(19) United States
(12) Patent Application Publication
(10) Pub. No.: US 2024/0399020 A1
(43) Pub. Date: Dec. 5, 2024

(54) METHODS AND MATERIAL COMPOSITIONS FOR PREVENTING NON-TARGET EMBOLIZATION

(71) Applicant: PneumoNix Medical, Inc., Andover, MA (US)

(72) Inventors: Joseph White, Woburn, MA (US); Anuj Bellare, Woburn, MA (US); James Wilkie, Andover, MA (US)

(73) Assignee: PneumoNix Medical, Inc., Andover, MA (US)

(21) Appl. No.: 18/675,318

(22) Filed: May 28, 2024

Related U.S. Application Data
(60) Provisional application No. 63/469,938, filed on May 31, 2023.

(51) Int. Cl. A61L 24/04

(52) U.S. Cl. CPC A61L 24/043; A61L 2400/04; A61L 2430/36

(57) ABSTRACT

The disclosure relates to devices, method, and material compositions. Specifically, the present invention related to a delivery device to deliver a material composition that prevents complications during minimally invasive procedures.

### RELATED APPLICATIONS

This application claims priority to U.S. provisional application Ser. No. 63/469,938 filed on May 31, 2023, which is incorporated into this disclosure in its entirety.

### FIELD OF THE DISCLOSURE

The disclosure relates to devices, method, and material compositions. Specifically, the present invention related to a delivery device to deliver a material composition that prevents complications during minimally invasive procedures. A foremost example includes preventing non-target embolization during embolization procedures.

### BACKGROUND INFORMATION

### Liver Cancer

There are about 40,000 new cases of liver cancer in the U.S. per annum with an average 5-year survival rate of 20%. There is both primary and secondary liver cancer. Primary liver cancer originates in the liver while secondary liver cancer (also known as metastatic liver cancer) originated from elsewhere in the body such as the stomach, breast, lung, pancreas, colon, etc. The most common form of liver cancer is hepatocellular carcinoma (HCC); however, other forms include intrahepatic cholangiocarcinoma (bile duct cancer), angiosarcoma and hemangiosarcoma (cancer of the blood cells that line the liver), and hepatoblastoma (cancer of fetal liver cells that commonly affects children younger than 4).

The liver is located in the upper-right hand portion of the abdominal kidney, beneath the diaphragm and on top of the stomach, right kidney, and intestines. The liver contains two crucial paths for blood supply in which oxygenated blood flows in from the hepatic artery and nutrient-rich blood flows in from the hepatic portal vein. In total the liver has two lobes which each contain eight segments consisting of 1,000 lobules (small lobes). The lobules are interconnected to small ducts which in total form the common hepatic duct which transports bile made by liver cells to the gallbladder and duodenum via the common bile duct.

The liver plays several important physiologic functions including but not limited to the production of: bile, proteins for blood plasma, cholesterol and proteins to carry fats through the body. Once the liver has broken down harmful substances, its by-products are excreted into the bile or blood. Bile products enter the intestine and leave the body in the form of feces while blood products are filtered out through the kidneys and leave the body in the form of urine.

Most normal liver cells are fed by the portal vein whereas in liver cancer the tumor is mainly fed by the hepatic artery. It has been established that blocking part of the hepatic artery which “feeds” the tumor assists with killing the cancerous cells while sacrificing the healthy liver cells which are still supplied with blood via the portal vein.

Embolization of liver cancer, sometimes known as transarterial embolization (TAE) or transarterial chemoembolization (TACE) or drug-eluting bead chemoembolization (DEB-TACE) or transarterial radioembolization (TARE), act to disrupt the blood supply of the tumor by block part of the hepatic artery. Common embolization particles used during TACE include PVA particles such as Bearing nsPVA™ (Merit Medical, South Jordan, UT), Contour™ PVA Embolization Particles (Boston Scientific, Marlborough, MA). During a TACE procedure the chemo agent is directed towards the target site and a bland embolization particle such as PVA is then placed in the target vessel to restrict blood flow and prevent reflux of the chemo agent. Common embolization particles for DEB-TACE include the DC Bead™ (Boston Scientific, Marlborough, MA) which can be loaded with a chemo agent such as doxorubicin thereby providing a more targeted chemotherapy. Common embolization particles for TARE include TheraSphere™ (Boston Scientific, Marlborough, MA) and SIR-Spheres® (Sirtex, Woburn, MA), typically these embolic particles are radioactive via Yttrium-90 (Y-90).

Additionally, some of these therapies such as TACE, DEB-TACE, and TARE have additional properties such as cytotoxicity to amplify the cancer killing properties in addition to disrupting the blood supply. Examples of chemotherapy drugs used during embolization procedures include but are not limited to mitomycin C, cisplatin, and doxorubicin. The chemotherapy drugs can be impregnated onto the embolization particles or the particles can contain radioactive properties (e.g., yttrium-90, also known as Y-90). Further, TACE, DEB-TACE, and TARE are not limited to only the liver, they can also be used on other organs including but not limited to the prostate, brain, stomach, lungs, kidney, pancreas and uterus.

There are numerous modalities to treat liver cancer such as ablation, surgery, chemotherapy, embolization, and even immune regulating therapies. However, embolization provides a minimally invasive option for patients with tumors that cannot be removed by surgery or for tumors that are too large to be treated by ablation. Further, embolization can be used concurrently with other therapies such as ablation. During an embolization procedure, a catheter is usually directed percutaneously through the femoral artery, iliac artery, abdominal aorta and into the hepatic arteries and ultimately the target site.

The goal of embolization for liver cancer is to effectively block the oxygen and blood supply to the tumor. However, due to the complex anatomy and tortuousity of vessels within the liver it can be challenging to accurately direct the embolization particles to the target. It is therefore the goal of the interventionalist to contain the embolization particles to the target vessel and/or lesion while reducing deposition of embolization particles in non-target vessels and/or tissues. This objective is further heightened upon using chemoactive or radioactive particles due to their heightened cytotoxic effects which can cause deleterious effects in non-target tissues and vessels. Further, it is important to direct as much of the chemoactive or radioactive particles into the target to provide a greater cancer killing effect.

Common side-effects of non-target embolization (also known as post-embolization syndrome) include transient abdominal pain, nausea, fever, ileus, and sometimes tissue death.

Current methods to prevent non-target embolization include diligent pre-procedural and intra-procedural planning such as using fluoroscopic and CT-guidance to better map vessel flow and vessel dynamics. Typically, the interventionalist will inject a contrast or dye such as Omnipaque (GE Healthcare, Chicago, IL) into the hepatic arteries and visualize the flow to gain a better understanding of the vessel dynamics such as flow direction, flow rate, and high/low pressure zones. The contrast acts as proxy for how the embolization particles will behave once injected.

Another method to prevent non-target embolization includes the use of specialized catheters such as the Sniper Balloon Catheter® (Embolx, Sunnyvale, CA) or the SeQure Reflux Control Microcatheters® (Guerbet, Villepinte, FR). However, the use of these catheters can require multiple access points which can crowd the number of devices that can traverse the hepatic vessels and can make it tricky to access very distal and small hepatic vessels.

Other methods include the prophylactic placement of coils or Gelfoam® (Pfizer, New York, NY). Endovascular coils such as Concerto Helix® (Medtronic, Minneapolis, MN) are typically made of a nitinol or similar like metal that coils upon itself upon detachment from the catheter. Blood cells aggregate onto the coil until a permanent block is created. However, the permanent nature of coils can be deleterious if it is blocking blood flow to a critical tissue or organ. Gelfoam is another material that is used to temporarily embolize a vessel. Gelfoam is a sterile compressed sponge made of purified porcine skin and acts as a natural hemostatic agent. Gelfoam has a non-uniform degradation profile depending on several factors such as the degree of saturation with blood or other fluids and the site of use. Gelfoam is well known to travel downstream from the application site which can cause non-target embolization. Additionally, Gelfoam has massive swelling properties which can have deleterious swelling consequences.

Common targets to protect from non-target embolization during liver embolization procedures include but are not limited to the cystic artery (CA), gastroduodenal artery (GDA), gastric artery (GA), and collateral hepatic arteries. One will recognize that it may be preferential to block additional vessels and the above illustrations are not meant to be limiting but rather examples of potential applications.

Another aspect of the present invention relates to both prostate artery embolization (PAE), and uterine fibroid embolization (UFE).

### Prostate Artery Embolization (PAE)

Another organ that undergoes embolization procedures is the prostate, typically to treat benign prostate hyperplasia (BPH). BPH is characterized by an enlargement of the prostate gland which can result in decreased urinary flow. Common treatments for BPH include surgical resection, drug therapy (e.g., alpha blockers or 5-alpha reductase inhibitors), ablation (e.g microwave, RF, lasor, vapor), robotic assisted surgery, and embolization.

Embolization of the prostate results in decreased blood supply to the prostate gland resulting in organ shrinkage. Similar to liver embolization procedures there are many collateral vessels which can lead to important tissues such as the rectal and penile tissues. It was reported in one study that 26% of patients undergoing PAE required prophylactic coiling of critical vessels (i.e., vessels that lead to the rectum or penis) (Naidu et al. Prostate Artery Embolization-Review of Indications, Patient Selection, Techniques and Results. J Clin Med. 2021 Oct. 31; 10 (21): 5139). Therefore, it is the goal of the interventionalist to prevent non-target embolization of these critical structures. Complications of this procedure may include dysuria, urinary infection, hematuria, hematospermia, acute urinary retention, and rectal bleeding.

During PAE a catheter is inserted via the femoral artery and is directed through a series of arteries including but not limited to: anterior division, posterior division, superior gluteal artery, superior vesical artery, obturator artery, prostatic artery, inferior gluteal artery, internal pudendal artery.

An emerging treatment is the utilization of PAE for prostate cancer (see Mouli, et al. (2021). Yttrium-90 Radioembolization to the Prostate Gland: Proof of Concept in a Canine Model and Clinical Translation. Journal of Vascular and Interventional Radiology, 32 (8)). During this procedure it is crucial to avoid non-target embolization of surrounding critical structures and ensure a high deposition of the radioactive or chemoactive drug within the target tissue.

### Uterine Fibroid Embolization (UFE)

Uterine fibroids (also known as leiomyomas or myomas) are growth that appear in the uterus that are made of uterine muscle. Although it is rare that fibroids are cancerous, they can have significant side-effects such as heavy menstrual bleeding, pelvic pressure, pelvic pain, frequent urination, backaches, and leg pains.

UFE works in a similar manner to PAE in that the embolic particles limit the blood flow to the fibroid essentially starving the fibroid of nutrients thereby shrinking the fibroid. During this procedure a catheter is inserted into the femoral artery and directed to the uterine arteries that lead to the fibroid target. It is the goal of the interventionalist to avoid non-target embolization of collateral vessels which lead to healthy tissue such as healthy uterine tissue.

The present invention relates to temperature sensitive polymers such as poloxamers, also known as pluronics or tri-block polymers and their utilization during medical procedures such as TACE, DEB-TACE, and TARE. Poloxamers have a tri-block copolymer structure of hydrophilic polyethylene oxide (PEO) end blocks and a central hydrophobic polypropylene oxide (PPO) block resulting in a temperature sensitive polymer. At low temperatures, a mixture of poloxamer and water exists in the solution state and as the temperature of the solution is raised, a micellular structure is thermodynamically favored. As temperatures rises, the association of the poloxamer micelles serve to physically crosslink the polymer by forming a crystalline structure thereby resulting in gelation. Poloxamers have an established history in implantable medical devices such as temporary endovascular embolics (see U.S. Patent 2005/0008610). These polymers can be delivered via a delivery device such as a catheter or microcatheter. Additionally, pluronics can be combined with other excipients such as polysaccharides, viscosity modifiers, fatty acids and others to optimize their function. Further, the polymers can occlude multiple vessels at a time, temporarily block blood flow, have uniform degradation, prevent non-target embolization, and have a safe profile upon migrating downstream.

It is an object of this disclosure to provide devices and methods for controlling fluid flow during a surgical procedure, more preferably preventing non-target embolization during an embolization procedure such as TACE, DEB-TACE, and TARE. The invention generally includes the use of a delivery device used to deliver a material including but not limited to hydrogels, sealants, polymers, and thermosensitive polymers. A thermosensitive polymer is a polymer that can change from a liquid-gel state dependent on a specific temperature or a range of temperatures. Using the devices and methods disclosed herein, the thermosensitive polymer can be delivered inside the body in a liquid form at which it will transform to the gel state or it can be delivered in a gel state in which it will stay in a gel state inside the body. Other advantages will be apparent from this disclosure to those of ordinary skill in the art.

### SUMMARY OF THE DISCLOSURE

This disclosure relates to devices, compositions and methods for preventing non-target embolization. In preferred embodiments, this disclosure provides methods (as well as devices and compositions used in such methods) for preventing non-target embolization comprising the steps of: (a) identifying a target in an organ such as the liver, prostate, uterus, or brain (b) advancing a delivery device towards the target (c) administering a first composition comprising a polymer to act as a sealant, preferably a thermosensitive polymer, to a location before or after reaching the target site wherein application of the polymer occurs in a location adjacent to the target (d) further advancing the delivery device to the target site or removing the delivery device; and, (e) performing a surgical step at the surgical site wherein the surgical step includes but is not limited to (TACE, DEB-TACE, TARE, tissue excision, ablation, embolization) (f) wherein the polymer prevents flow of a biologic fluid throughout the surgical step and optionally a longer time period, more specifically the polymers and/or thermosensitive polymers act to prevent the flow of blood to reduce the risk of non-target embolization.

In certain embodiments, this disclosure relates to the aforementioned methods, wherein said thermosensitive is a block polymer or a branched copolymer.

In certain embodiments, this disclosure relates to the aforementioned methods, wherein said thermosensitive polymer is an optionally purified poloxamer, poloxamine, or pluronic.

In certain embodiments, this disclosure relates to the aforementioned methods, wherein said thermosensitive polymer is optionally purified and selected from the group consisting of poloxamine 1107, poloxamine 1307, poloxamer 338 and poloxamer 407.

In certain embodiments, this disclosure relates to the aforementioned methods, wherein said thermosensitive polymer solution has a transition temperature of between about 5° C. and 40° C.

In certain embodiments, this disclosure relates to the aforementioned methods, wherein the first composition further comprises at least one fatty acid.

In certain embodiments, this disclosure relates to the aforementioned methods, wherein the adjacent location can be interpreted as the cystic artery (CA), gastroduodenal artery (GDA), gastric artery (GA), collateral hepatic arteries, collateral prostatic arteries, collateral uterine arteries, and collateral arteries within the brain. Artisans within the art will appreciate that other vessels not mentioned herein have been contemplated and appreciated. Specifically, the present invention contemplates and appreciates vessels that are collateral to the target.

In certain embodiments, this disclosure relates to the aforementioned methods, wherein the first composition is administered to a site that is located proximal, distal, or adjacent the target. It has been recognized that the first composition can be administered via a delivery device at a location proximal to the target wherein after application of the first composition the delivery device traverses through the first composition to a location distal to the first composition. It has been recognized that the first composition can be applied at a location adjacent to the target site such as in a collateral vessel wherein after application of the first composition the delivery device is redirected to the target which may be proximal, distal, or adjacent of the first composition.

In certain embodiments, this disclosure relates to the aforementioned methods, wherein the target is selected from the group consisting of but not limited to a liver, prostate, uterus, lung, brain, kidney, liver, spleen, lesion, hemorrhage, tumor, dural sac, cancerous tissue, organ, aneurysm, arteriovenous malformation, and arteriovenous fistula.

In certain embodiments, this disclosure relates to the aforementioned methods, wherein the surgical step or surgical procedure is selected from the group consisting of but not limited to TACE, DEB-TACE, TARE, tissue excision, ablation, embolization, particle embolization, liquid embolization.

In certain embodiments, t this disclosure relates to the aforementioned methods, wherein said first composition is injected via a delivery device wherein the delivery device includes but is not limited to a catheter, microcatheter, guide-catheter, syringe, and power-injector. Wherein the syringe has a volume capacity between 0.25 mL to 10 mL or 1 mL to 3 mL. Artisans within the art will appreciated that any volume within these bounds and volumes outside of these bounds have been contemplated and appreciated.

In certain embodiments, this disclosure relates to the aforementioned methods, wherein said first composition is injected via a delivery device wherein the delivery device has a diameter between 0.1 French and 10 French or 0.1 French and 3 French. Artisans within the art will appreciated that any diameter within these bounds and diameters outside of these bounds have been contemplated and appreciated.

In certain embodiments, this disclosure relates to the aforementioned methods, wherein the first composition comprises a thermosensitive polymer that is administered in a gel state. Artisans within the art will appreciate that due to the thermosensitive nature the first composition can undergo a liquid-gel, liquid-liquid, gel-liquid, or gel-gel transition depending on the temperature that the first composition is exposed to, all of these transformations have been contemplated and appreciated.

In certain embodiments, this disclosure relates to the aforementioned methods, wherein the first composition when administered in the body and preferably proximal, distal, or adjacent to a target. Wherein the first composition helps to prevent the flow of biologic fluid and embolic particles.

In certain embodiments, this disclosure relates to the aforementioned methods, wherein said first composition comprises about 5% to 45% or 10% to 40% of said thermosensitive polymer.

In certain embodiments, this disclosure relates to the aforementioned methods, wherein the thermosensitive polymer further comprises a contrast-enhancing agent and/or a tissue dye material.

In certain embodiments, this disclosure relates to the aforementioned methods, wherein said contrast-enhancing agent is selected from the group consisting of radiopaque materials, heavy atoms, transition metals, dyes, and radionuclide-containing materials. Examples include iohexol (Omnipaque®, GE Healthcare, Chicago, IL) or gadolinium (Gadavist®, Bayer, Leverkusen, DE).

In certain embodiments, this disclosure relates to the aforementioned methods, wherein the composition comprises about 5% to 45% or 10% to 40% of said thermosensitive polymer and between about 0% to 25% contrast-enhancing agent. Furthermore, a dye can also be included in the composition.

In certain embodiments, this disclosure relates to the aforementioned methods, wherein the composition comprises 0% to 40% of an excipient such as polysaccharides (starches, gluycogens, and cellulose). More specifically, the polysaccharides can include but is not limited to hyaluronic acid, alginate, chitosan, carboxymethylcellulose, hydroxypropyl methylcellulose, sucrose, dextran, agarose. Further, the composition can comprise viscosity modifiers including but not limited to carbomers, Carbopol® (Lubrizol, Wickliffe, OH), polymethacrylate (PMA), and polyacrylic acid (PAA). Further viscosity modifiers include glycerol, oils, lipids, fatty acids, proteins, carbohydrate-based polymers, and synthetic polymeric materials commonly used as pharmaceutical excipients.

In certain embodiments, this disclosure relates to the aforementioned methods, wherein the composition can comprise hyaluronic acid, chitins, chitosans, and alginates, as well as polypeptides and polysaccharides like starch and dextran. Proteins including albumins, collagens, and gelatins can be used as crosslinkers with various polymers to form a suitable gel with a viscosity greater than water. Protein crosslinkers for further processing of natural hydrogels include but are not limited to aldehydes such as gluteraldehyde, other polyaldehydes, and esters. Proteins can be derived from either natural, semi-synthetic, or synthetic processes. Synthetic hydrogels do not generally biodegrade and can be comprised of polymers such as poly(hydroxylalkyl methacrylates), polyacrylamide (PAM), polyvinylpyrrolidone (PVP), polyvinyl alcohol (PVA), polyglycolides including polyglycolic acid (PGA), polyactides including polylactic acid (PLA), polylactic-co-glycolic acid (PLGA), polycaprolactone (PCL), polydioxanone (PDO), poly(ε-caprolactone-co-glycolic acid) (PCGA), Poly(N-isopropylacrylamide) (PNiPAam), polyethylene glycol (PEG), polyethylene glycol diacrylate (PEGDA), polyethylene glycol dimethacrylate (PEGDMA), polyethylene oxide (PEO), polypropylene oxide (PPO), derivatives from triblock copolymers including to but not limited to PEO-PPO-PEO and PPO-PEO-PPO blocks, synthetic collagen, silicone, and synthetic gelatin. Furthermore, the sealant may in the form of a gel, liquid, or microsphere wherein the microsphere has a diameter in the range of 20-500 microns, or in the range of 1-100 nm. Biodegradability of the sealant composition can be increased by adding monomers from the groups including but not limited to glycolide, lactide, ε-caprolactone, p-Dioxanone, and Trimethylene Carbonateln. The hydrogels described above could be formulated to swell and expand in the presence of aqueous fluid (i.e. biological fluid), and be activated (i.e. change physical and chemical properties) upon exposure to pH, fluid, blood, saline, temperature, light, electron-beam, gamma-radiation, UV, DNA, enzymes, and other suitable initiators.

Additionally, in certain embodiments, the methods disclosed herein may be used to deliver a therapeutic substance such as anthistamines, analgesics, immunosupressive agents, coronary, cerebral or peripheral vasodilators, hormonal agents, antithrombotic agents, diuretics, antihypertensive agents, cardiovascular drugs, opioids or a combination of those thereof. Preferred therapeutic agents are those directed towards diseases including but not limited to liver cancer, BPH, and uterine fibroids. The amount of therapeutic agent to be delivered will be dependent on the disease state and can be varied over time.

Other embodiments are also disclosed herein as will be apparent those of ordinary skill in the art.

### BRIEF DESCRIPTION OF THE DRAWINGS

FIG. 1a illustrates a device adapted to deploy a first composition in the liver.

FIG. 1b illustrates deposition of a first composition in the liver.

FIG. 2 illustrates deposition of a first composition in the prostate.

FIG. 3a illustrates a device adapted to deploy a first composition in a vessel.

FIG. 3b illustrates deposition of a first composition in a vessel.

FIG. 3c illustrates reconfiguration of a device after deploying a first composition in a vessel.

### DETAILED DESCRIPTION

As used herein, the singular forms “a”, “an”, and “the” refer to one or more than one, unless the context clearly dictates otherwise.

As will be appreciated by persons skilled in the art, the invention and its embodiments have been described with respect to procedures involving the liver, prostate, and uterus. However, certain aspects of the device and method are applicable to other procedures and devices suitable elsewhere in the body. These may include but are not limited to kidney, brain, stomach, bladder, joints, pancreas, muscle and gastrointestinal tract. Treatment modalities include but are not limited to redirecting blood flow, preventing non-target embolization, and enhancing drug delivery to a target. Furthermore, the location of implantation of the polymer may be described as being proximal, distal, or adjacent to a site. For example, in the context of a TACE procedure the polymer may be deployed in a vessel that is proximal to the target. In this context, proximal is defined as the delivery device, preferably a catheter or microcatheter, must travel past the proximal vessel before reaching the target. Contrarily, distal is defined as the delivery device, preferably a catheter or microcatheter, must travel past the target before reaching the target. Further, adjacent is defined as a vessel that is within a similar plane of the target or is part of a separate bundle or branch of vessels.

As used herein, the term “subject” can include a living human subject, cadaver, swine model, canine model, rabbit model, mouse model, or rat model.

As used herein, the term “tissue” can include any tissue within the body. The present invention focuses on the liver, prostate, and uterus. However, the device can also relate to different organs including but not limited to kidney, brain, stomach, bladder, joints, pancreas, muscle and gastrointestinal tract.

As used herein, the term “target” is broad and may refer to a cancerous lesion, suspicious lesion, site within an organ, fissure, hemorrhage, arteriovenous malformation, and arteriovenous fistula.

As used herein, the term “polymer” means a molecule, formed by the chemical union of two or more oligomer units. The chemical units are normally linked together by covalent linkages. The two or more combining units in a polymer can be all the same, in which case the polymer is referred to as a homopolymer. They can be also be different and, thus, the polymer will be a combination of the different units. These polymers are referred to as copolymers. The term “polymer” herein may be also be interchanged with the terms sealant or hydrogel.

The thermosensitive polymer described herein may also be referred to as “inverse thermosensitive” or “inverse gelling” or “reversibly gelling” which refers to the gelation occurring upon an increase in temperature rather than a decrease in temperature. The temperature at which the polymer transforms to a gel may be referred to as the “transition temperature”.

The term “contrast-enhancing” refers to materials capable of being monitored during injection into a subject by methods of monitoring for detecting such materials for example by CT-Scan, X-Ray, MRI, fluoroscopy, and ultrasound. An example of a contrast-enhancing agent is a radiopaque material. Contrast-enhancing agents including radiopaque materials may be water soluble or water insoluble. Examples of water-soluble radiopaque materials include metrizamide, iopamidol, iothalamate sodium, iohexol, iodomide sodium, gadolinium, and meglumine. Examples of water insoluble radiopaque materials include metals and metal oxides such as gold, titanium, silver, stainless steel, nitinol, oxides thereof, aluminum oxide, zirconium oxide, and compounds with similar properties and uses as would be understood by those of ordinary skill in the art.

The term “biocompatible”, as used herein, refers to having the property of being biologically compatible by not producing a toxic, injurious, or immunological response in living tissue.

The terms “poloxamer” and “pluronic” denote a symmetrical block copolymer, consisting of a core of PPG polyoxylethylated to both its terminal hydroxyl groups, i.e., conforming to the interchangeable generic formula (PEG)_{X}-(PPG)_{Y}-(PEG)_{X} and (PEO)_{X}-(PPO)_{Y}-(PEO)_{X}. Each poloxamer name ends with an arbitrary code number, which is related to the average numerical values of the respective monomer units denoted by X and Y.

The term “collateral vessels”, as used herein, refers to vessels that are connected to a target. For example, the liver may contain a tumor or mass therein which sources blood flow from a number of vessels which may be referred to as collateral vessels. The present invention also appreciates that collateral vessels are found in other areas of the body including but not limited to the prostate, uterus, brain, kidney, and lung.

Examples of thermosensitive polymers described herein include poloxamer 4078, poloxamer 188, Pluronic® F127, Pluronic® F68, poly(N-isopropylacrylamide), poly(methyl vinyl ether), poly(N-vinylcaprolactam); and certain poly(organophosphazenes) (see Bull. Korean Chem. Soc. 2002, 23, 549-554). As used herein, the terms Poloxamer 407 and Pluronic® 127 are used interchangeably and refer to the same polymer. Further, Poloxamer 407 and/or Pluronic® 188 can be used.

In preferred embodiments, this disclosure relate to devices, reagents and methods providing one with the ability to deploy a polymer in blood vessels to redirect blood flow to a target, and in some preferred embodiments, reduce complications related to non-target embolization. The devices and methods are used with polymer compounds which have a suitable, density, viscosity, modulus, degradation profile and other material properties to effectively prevent non-target embolization. In some embodiments it may be preferential to have the material degrade after a specific time period such as >1 month, 2-4 weeks, 1-2 weeks, 1 day-3 days, 1 hour to 24 hours, and more preferably between 1 hour and 3 hours. Thus, in some embodiments, this disclosure provides a device that can be used to administer, deposit, implant, or deploy a polymer into a vessel such that biologic fluid and/or embolization particles cannot enter the vessel for a specific period of time. In some preferred embodiments, this disclosure relates to the degradation of the polymer wherein the polymer degrades within a specific time-period after administration, deposition implantation, and/or deployment.

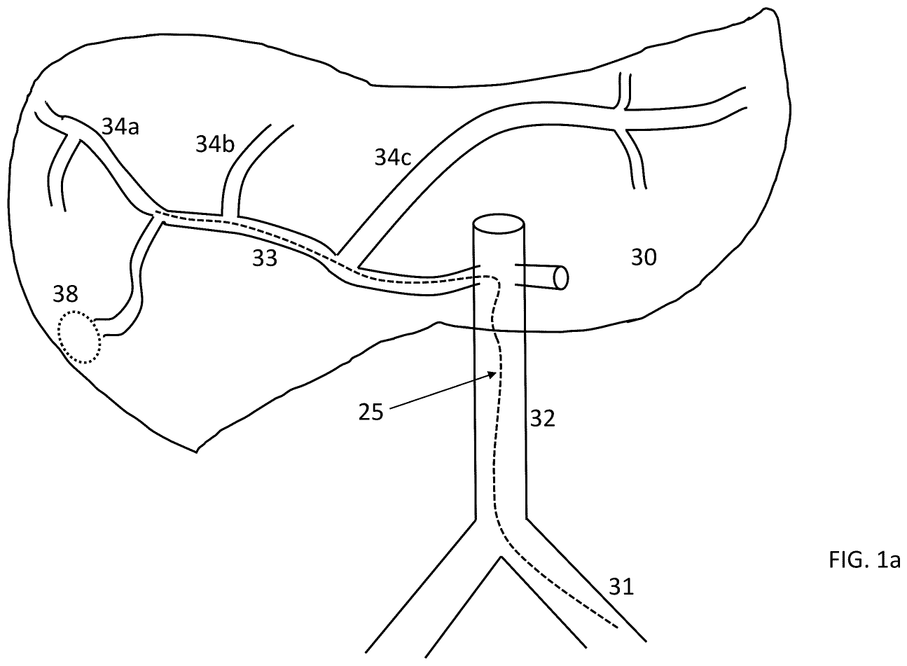

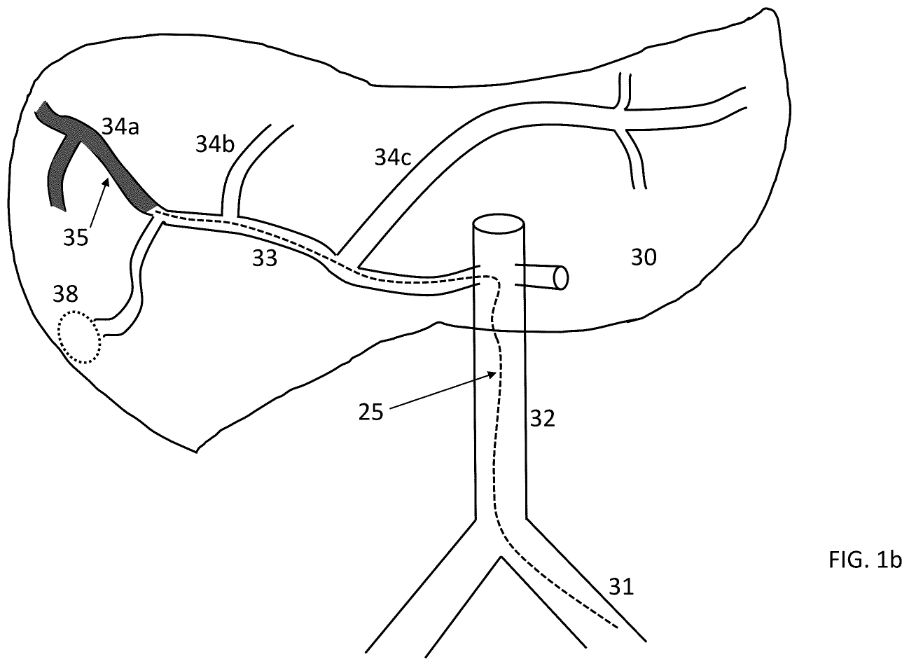

FIG. 1a portrays an exemplary delivery device 25 being directed through a iliac artery 31 and aorta 32 into a liver 30 and, more specifically, a hepatic artery 33. There are numerous procedures which following this anatomical roadmap including but not limited to TACE, DEB-TACE, TARE. The goal during the procedure is to restrict blood flow in the vessel that is feeding target 38. The target 38 can represent, for instance, a lesion and/or tumor. A key risk when trying to embolize the feeder vessel relates to embolization particles traveling to other hepatic collateral vessels 34a, 34b, and 34c (i.e., that are not intended to be blocked).

The delivery device 25 is typically a microcatheter with a distal tip diameter between 2 French and 4 French. Further the delivery device 25 will typically have a lumen size between 0.021″ and 0.05″. Further, the delivery device 25 may be between 100 and 200 cm and may have multiple tip shapes such as a straight, “J”, or swan which are understood by one of ordinary skill in the art. Further, the delivery device 25 may be comprised of a PEEK or other polymer material which can with stand injection pressures between 1,000 to 5,000 PSI. Further, the delivery device 25 may include a hydrophilic coating such as poly(vinyl alcohol) or poly(acrylic acid).

FIG. 1b portrays a preferred embodiment of a polymer 35 deployed into the hepatic collateral vessel 34a, polymer 35 being represented as the shaded interior of collateral vessel 34a. In some embodiments, it may be advantageous to deploy the polymer 35 in this location due to the risk of non-target embolization, flow dynamics, or to enhance deposition of embolization particles towards target 38. For example, hepatic collateral vessel 34a may lead to another organ or healthy tissue which would have deleterious effects if embolized. Further, vessel 34a may represent an area of higher flow or pressure that may redirect embolization particles away from target 38. Further, blocking hepatic collateral vessel 34a may allow increased deposition of the embolization particles towards target 38 thereby increasing the efficacy of the TACE, DEB-TACE, or TARE procedure. FIG. 1b merely provides one example of the present invention it may be advantageous in some cases to deploy polymer 35 in all or part of other hepatic collateral vessels in addition to 34a such as 34b and 34c. Further, polymer 35 may be deployed before, during, or after deployment of the embolization particles. For example, it may be advantageous to deploy polymer 35 into the feeder vessel after embolization particles have been directed to the target 38 to prevent reflux of the therapeutic embolization particles. Further, the polymer 35 may be tuned to degrade within a specific time-period after deployment such as between 1 day-3 days, 1 hour to 24 hours, and more preferably between 1 hour and 3 hours.

It may be preferable to prophylactically deploy polymer 35 in the cystic artery (CA), gastroduodenal artery (GDA), gastric artery (GA) to prevent non-target embolization of critical structures such as healthy tissues and organs.

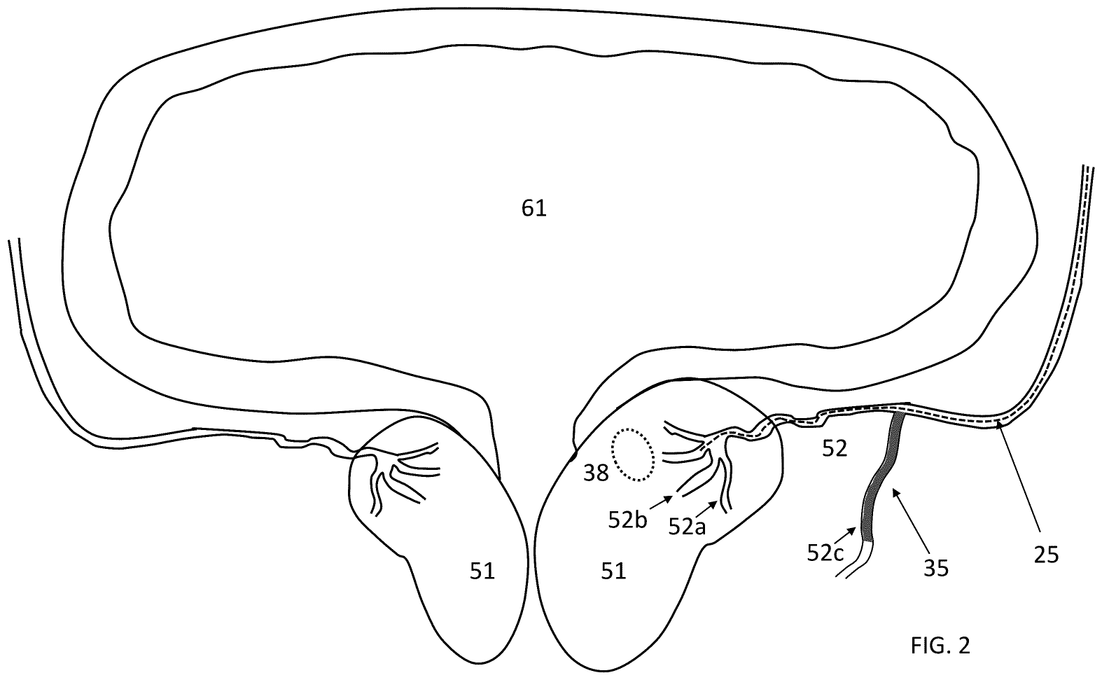

FIG. 2 illustrates another preferred embodiment relating to the prostate gland 51. In this exemplary preferred embodiment, delivery device 25 is directed through the prostate artery 52 to deploy the polymer 35 into the prostatic collateral vessels 52a, 52b, or 52c to prevent non-target embolization or to increase deposition of a therapeutic agent into the target. Deposition into prostatic collateral vessel 52c is indicated in FIG. 2 as the shaded portion of that vessel. Further, prostatic collateral vessels (e.g., 52a, 52b, or 52c) may lead towards critical structures such as penile or rectal tissue, the embolization (i.e., blockage) of which can cause deleterious effects. Such prostatic collateral vessels (e.g., 52a, 52b, and/or 52c) can be embolized before, during, or after delivery of a therapeutic agent to the target 38. In some embodiments, the target may represent the entire prostate gland 51 or may represent a lesion, tumor, and/or mass.

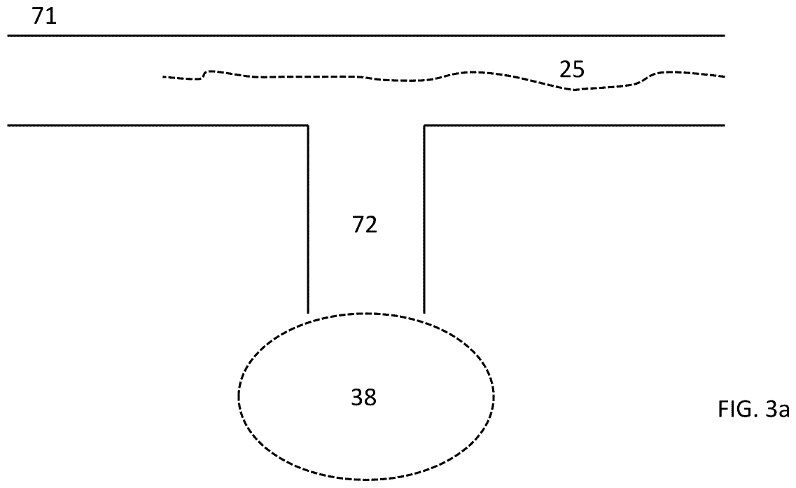

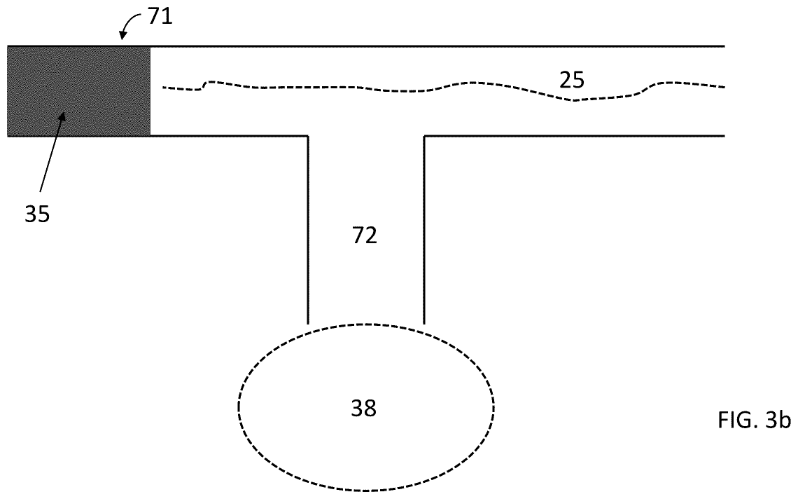

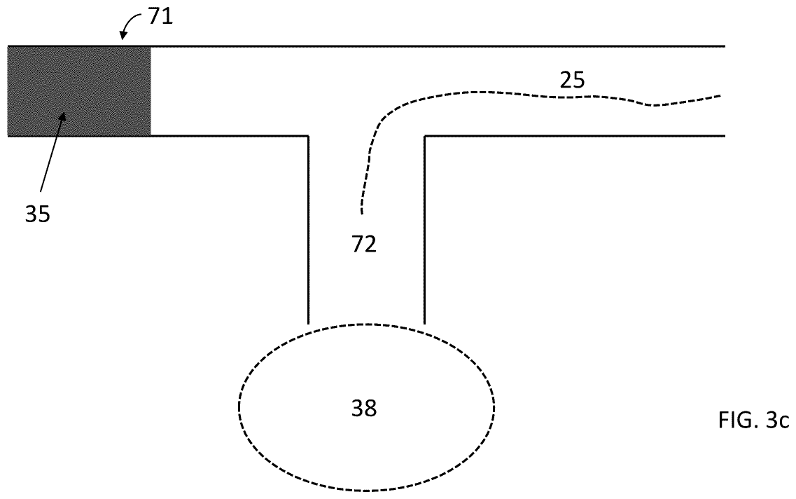

FIGS. 3a-3c illustrate and exemplary preferred embodiment in which delivery device 25 can be directed to a collateral vessel 71 and polymer 35 deployed (see the shaded area in FIG. 3b). The delivery device can then be redirected into target vessel 72 (FIG. 3c) to deploy embolization particles or a therapy such as one or more anti-cancer or immunomodulation drugs toward target 38. Polymer 35 may be deployed in a plurality of collateral vessels that may or may not feed the target vessel 72. Further, polymer 35 may be deployed before, during, or after the treatment on target 38 is performed. The treatment on target 38 may include but is not limited to deployment of embolization particles, deployment of chemotherapy, deployment of radioactive particles, cryoablation, RF ablation, microwave ablation, excision, biopsy, and minimally invasive excision.

The embodiments illustrated in FIGS. 3a-3c are not meant to be limiting but rather explain the breadth of locations and manners that deployment of polymer 35 can be used. For example, polymer 35 may be deployed proximal to or adjacent to target vessel 72. Further, polymer 35 may be deployed in a vessel that does not interconnect with target vessel 72.

Polymer 35 may contain radiopaque materials, heavy atoms, transition metals, dyes, and radionuclide-containing materials. Examples include iohexol (Omnipaque®, GE Healthcare, Chicago, IL) or gadolinium (Gadavist®, Bayer, Leverkusen, DE). Radiopacity of the polymer 35 may aid the user in accurate deployment via fluoroscopic, CT, or MRI imaging.

Polymer 35 may be deployed via a syringe containing a volume between 10 ml and 100 ml or 1 ml and 10 ml such as the Medallion® Syringe (Merit Medical, South Jordan, UT). Further, the polymer 35 may be deployed via a high-pressure syringe injector such as VacLok® (Merit Medical, South Jordan, UT).

### Polymer Formulations

Poloxamers, also known as Pluronics®, have unique surfactant abilities and extremely low toxicity and immunogenic responses. Pluronic® polymers are among a small number of surfactants that have been approved by the FDA for direct use in medical applications (Pluromed, Woburn, MA) and (BASF (1990) Pluronic® & Tectronic® Surfactants, BASF Co., Mount Olive N.J.). Poloxamers as nonionic surfactants are widely used in industrial applications in which their surfactant properties are useful in detergents, dispersion, stabilization, foaming, and emulsification.

Several Poloxamers show inverse thermosensitivity within physiologic temperature ranges (e.g., poloxamer 188, poloxamer 407, poloxamer 338, poloxamine 1107, and poloxamine 1307). In other words, these polymers are soluble in aqueous solutions at low temperatures but gel at higher temperatures. Poloxamer 407 is a biocompatible polyoxypropylene-polyoxyethylene block copolymer having an average molecular weight of about 12,500 and a polyoxypropylene fraction of about 30%; poloxamer 188 has an average molecular weight of about 8400 and a polyoxypropylene fraction of about 20%; poloxamer 338 has an average molecular weight of about 14,600 and a polyoxypropylene fraction of about 20%; poloxamine 1,107 has an average molecular weight of about 14,000, poloxamine 1307 has an average molecular weight of about 18,000. Polymers of this type are also referred to as reversibly gelling because their viscosity increases and decreases with an increase and decrease in temperature, respectively. Such reversibly gelling systems are useful wherever it is desirable to handle a material in a fluid state, but performance is preferably in a gelled or more viscous state. As noted above, certain poly(ethyleneoxide)/poly (propyleneoxide) block copolymers have these properties; they are available commercially as Pluronic® poloxamers and Tetronic® poloxamines (BASF, Ludwigshafen, Germany) and generically known as poloxamers and poloxamines, respectively. See U.S. Pat. Nos. 4,188,373, 4,478,822 and 4,474,751. Throughout the application terms Pluronic® 127 and Poloxamer 407 represent the same polymer from different manufacturers and are used interchangeably.

Other Poloxamers that are envisioned and considered include but are no limited to: P105, P108, P122, P123, P124, P182, P183, P184, P185, P188, P212, P215, P217, P234, P235, P237, P238, P288, P333, P334, P335, P338, P402, P403, P407. Additionally, their respective Pluronic® names include L35, F38, L42, L43, L44, L62, L63, L64, P65, F68, L72, P75, F77, P84, P85, F87, F88, F98, P103, P104, P105, F108, L122, P123, F127. The formulations listed in the applications herein may contain a singular or multiple combination of the Poloxamer and Pluronic polymers listed above.

The average molecular weights of poloxamers range from about 1,000 to 16,000 daltons. In addition, commercially available poloxamers contain substantial amounts of poly(oxyethylene) homopolymer and poly(oxyethylene)/poly(oxypropylene) diblock polymers. The relative amounts of these byproducts increase as the molecular weights of the component blocks of the poloxamer increase. Depending upon the manufacturer, these byproducts may constitute from about 15 to about 50% of the total mass of the polymer.

The thermosensitive polymers of the present invention are also suitable delivery vehicles for conventional small-molecule drugs as well as macromolecular drugs such as peptides. Therefore, the thermosensitive polymers may comprise a pharmaceutic or therapeutic agent. The polymers described are capable of solubilizing and releasing pharmaceutic or therapeutic agents. Solubilization will occur as a result of dissolution in the bulk aqueous phase or by incorporation of the solute in micelles created by the hydrophobic domains of the poloxamer. Release of the agent would occur through diffusion or network erosion means.

Preferably, the agent incorporated into the poloxamer material is water soluble which lends itself to a homogenous dispersion throughout the thermosensitive composition. The agent incorporated within the poloxamer material can be, for example, an anesthetic, antimicrobial, antifungal, antiviral, anti-inflammatory, diagnostic, wound killing, and/or cancer killing compound and/or combination.

The poloxamer may be combined with a wide array of pharmaceutical agents that may have biological activity, including proteins, polypeptides, polynucleotides, nucleoproteins, polysaccharides, glycoproteins, lipoproteins, and synthetic and biologically engineered analogs thereof.

The poloxamer may be combined with a wide array of therapeutic agents including but not limited to one or more anti-infectives such as antibiotics and antiviral agents; analgesics and analgesic combinations; anorexics; anti-helmintics; antiarthritics; anti-asthmatic agents; anticonvulsants; antidepressants; antidiuretic agents; antidiarrheals; antihistamines; anti-inflammatory agents; anti-migraine preparations; anti-nauseants; anti-neoplastics; anti-parkinsonism drugs; anti-pruritics; anti-psychotics; anti-pyretics, anti-spasmodics; anti-cholinergics; sympathomimetics; xanthine derivatives; cardiovascular preparations including calcium channel blockers and beta-blockers such as pindolol and anti-arrhythmics; antihypertensives; diuretics; vasodilators including general coronary, peripheral and cerebral; central nervous system stimulants; cough and cold preparations, including decongestants; hormones such as estradiol and other steroids, including corticosteroids; hypnotics; immunosuppressives; muscle relaxants; parasympatholytics; psychostimulants; sedatives; and tranquilizers; and naturally derived or genetically engineered proteins, polysaccharides, glycoproteins, or lipoproteins.

Additionally, the pharmaceutic agent combined with the poloxamer may be assigned to a certain class of agents; for example, including but not limited to anti-cancer substances, antibiotics, immunosuppressants (e.g., cyclosporine) anti-viral substances, enzyme inhibitors, neurotoxins, opioids, hypnotics, antihistamines, lubricants tranquilizers, anti-convulsants, muscle relaxants and anti-Parkinson substances, anti-spasmodics and muscle contractants, miotics and anti-cholinergics, anti-glaucoma compounds, anti-parasite and/or anti-protozoal compounds, anti-hypertensives, analgesics, anti-pyretics and anti-inflammatory agents such as NSAIDs, local anesthetics, ophthalmics, prostaglandins, anti-depressants, anti-psychotic substances, anti-emetics, imaging agents, specific targeting agents, neurotransmitters, proteins, cell response modifiers, and vaccines.

Anti-inflammatory agents may also be added to the thermosensitive polymers of the present invention including but not limited to propionic acid derivatives, acetic acid, fenamic acid derivatives, biphenylcarboxylic acid derivatives, oxicams, including but not limited to aspirin, acetaminophen, ibuprofen, naproxen, benoxaprofen, flurbiprofen, fenbufen, ketoprofen, indoprofen, pirprofen, carporfen, and bucloxic acid and the like.

Anesthetic agents may also be added to the thermosensitive polymers of the present invention including but not limited to bupivacaine, lidocaine, prilocaine, levobupivacaine, ropivacaine, mepivacaine, dibucaine, etidocaine, procaine, amethocaine, benzocaine, tetracaine, alfentanil, fentanyl, buprenorphine, butorphanol, diamorphine, hydromorphone, levorphanol, pethidine, methadone, nalbuphine, oxymorphone, pentazocine.

Further, the poloxamer or combination may be combined with other excipients such as viscosity modifiers, crosslinkers, and polysaccharides. Examples of viscosity modifiers include carbomers or Carbopol®. Carbomers consist of polyacrylic acid which is a derivative of acrylic acid. Examples of commercially available carbomers include Carbopol: 971NP, 934P NF, 934 NF, 940 NF, 941 NF, 1342 NF, 971P NF, 974P NF, 980 NF, and 981 NF. Examples of polysaccharides which may be used in the present invention include but are not limited to chitosan, hyaluronic acid, pectin, alginate, hydroxyethylcellulose, methylcellulose, sodium hyaluronate, sucrose, agar, carrageenan, and starch.

In certain embodiments, the present invention relates to the aforementioned method, wherein the composition comprises said thermosensitive polymer and a fatty-acid. In preferred embodiments, the thermosensitive polymer and fatty-acid are mixed creating an emulsion. In some preferred embodiments, the fatty-acid can comprise a carbon-chain consisting of between about 1-21 carbon atoms. In some preferred embodiments, the fatty acid can comprise a melting point below or above 37° C. In some embodiments, the fatty acid can comprise a carbon-chain consisting of between about 1-21 carbon atoms a melting point below or above 37° C. Examples of fatty acids described herein include myristic acid, stearic acid, capric acid, linoleic acid, lauric acid, palmitic acid, palmitoleic acid, arachidonic acid, behenic acid, lignoceric acid, cerotic acid, vaccenic acid, linoelaidic acid, eicosapentaenoic acid, erucic acid, docosahexaenoic acid, elaidic acid, gondoic acid, nervonic acid, mead acid, butyric acid, valeric acid, caproic acid, enanthic acid, caprylic acid, pelargonic acid, undecyclic acid, tridecylic acid, pentadecylic acid, margaric acid, heneicosylic acid, tricosylic acid, lignoceric acid, pentacosylic acid, carboceric acid, montanic acid, nonacosylic acid, melissic acid, hentriacontylic acid, lacceroic acid, psyllic acid, geddic acid, ceroplastic acid, hexatriacontylic acid, heptatriacontylic acid, octatriacontylic acid, nonatriacontylic acid, tetracontylic acid. The aforementioned list of fatty acids is not meant to be limiting but rather an example of the broad amount of fatty acids that have been considered. Compositions considered herein comprising a thermosensitive polymer and at least one fatty acid. Exemplary and preferred fatty acids can include myristic and palmitic acid. The preferred w/w ratio of fatty acids can be between about 5% and 20% and more preferably between about 10% and 20% w/w. Further it is preferred that the degradation time in-vivo and further investigated via the aggressive erosion test as described in Example 8 below is greater than 24 hours. Fatty acids can be classified by length, saturation (vs. unsaturation), carbon content, and linear (vs. branched). Further, fatty acids can be animal or plant derived. The applicant has herein considered short-chain, medium-chain, long-chain, and very-long-chain fatty acids. The applicant herein has considered fatty acids containing: 1-5 carbons, 6-12 carbons, 13-21 carbons, and above 22 carbons. The applicant herein has considered saturated and unsaturated fatty acids. Thermosensitive polymers such as poloxamer 407, poloxamer 188, Pluronic® F127, Pluronic® F68, poly(N-isopropylacrylamide), poly(methyl vinyl ether), poly(N-vinylcaprolactam); and certain poly(organophosphazenes) can be mixed with one or more of the fatty-acids listed above. Thermosensitive polymers such as poloxamer 407 and poloxamer 188 provide advantageous characteristics for in-vivo applications due to their gelation temperature which can range from about 10° C. to >37° C. Because the thermosensitive polymer gels at body temperature it can be advantageous to occupy a space in-vivo such as filling a void or defect and can also act as a delivery vehicle for a drug or therapeutic. Thermosensitive polymers are also recognized for their rapid degradation in-vivo (see Table 5 below). Therefore, in order to increase the degradation time of thermosensitive polymers it may be advantageous to add an excipient such as a fatty-acid. Some fatty acids including but not limited to myristic and palmitic acid have melting temperatures >37° C. This quality and their long carbon chains increase the degradation time of the composition when mixed with thermosensitive polymers. In preferred embodiments, the formulations comprise at least 10% Poloxamer 407 and/or at least about 10% Poloxamer 188 (preferably “or”); and at least 10% myristic and/or at least about 10% palmitic acid (preferably “or”). In some preferred embodiments, the formulation comprises at least about 30% of Poloxamer 188, at least about 10% palmitic acid, and 1×PBS. In such preferred embodiments, it is preferred that the degradation time as measured by the aggressive vial erosion test presented in Table 2 is at least about 24 hours or longer. When poloxamers and fatty acids are combined the fatty-acid in the crystalline state are less mobile than in their molten state thereby hindering the poloxamer ability to migrate to the free solvent. Further, the fatty-acid molecules can act as a barrier such that it is more difficult for the free solvent to penetrate between the fatty acid particles and thus liberate the poloxamer unimers. The inclusion of other fatty acids and/or combinations are also contemplated herein, as would be understood by those of ordinary skill in the art.

Thus, this disclosure provides reagents (e.g., medicaments), devices, and methods for use in the prevention of non-target embolization in a mammal by administering a first composition comprising a thermosensitive polymer to a group consisting of hepatic vessels, prostatic vessels, and/or uterine vessels. In preferred embodiments, the thermosensitive polymer is a block polymer, random copolymer, graft polymer or branched copolymer. In preferred embodiments, the first composition has a transition temperature between 5° C. and 40° C. In preferred embodiments, the first composition comprises 5% to 40% by weight, preferably 20% to 35% by weight of said thermosensitive polymer. In preferred embodiments, the first composition comprises 0.1% to 6% by weight of at least one excipient. In preferred embodiments, the the excipient is selected from the group consisting of polyacrylic acid, carbomer, Carbopol 934P NF, carboxymethylcellulose, sodium hyaluronate, hydroxypropyl methylcellulose, alginate, sucrose, starch, or agar. In preferred embodiments, the first composition comprises two or more excipients each consisting of 0.1% to 6% by weight. In preferred embodiments, the excipients are selected from the group consisting of polyacrylic acid, carbomer, Carbopol 934P NF, carboxymethylcellulose, sodium hyaluronate, hydroxypropyl methylcellulose, alginate, sucrose, starch, or agar. In preferred embodiments, the first composition comprises more than about 50% by weight of phosphate buffered saline. In preferred embodiments, the thermosensitive polymer is selected from the group consisting of poloxamine 1107, poloxamine 1307, poloxamer 338 poloxamer 407, and poloxamer 188. In especially preferred embodiments, the thermosensitive polymer is poloxamer 407. In preferred embodiments, the thermosensitive polymer is purified. In some preferred embodiments, the first composition can comprise at least one fatty acid. In some preferred embodiments, the fatty acid is selected from the group consisting of myristic acid, palmitic acid, stearic acid, linoleic acid, capric acid, lauric acid, and castor oil. In some especially preferred embodiments, the fatty acid is palmitic or myristic acid, and is most preferably myristic acid. In some preferred embodiments, the first composition comprises at least 10% w/w of Poloxamer 407 and at least 10% w/w of a fatty acid, preferably wherein the fatty acid is palmitic or myristic acid. In some preferred embodiments, the composition comprises at least about 10% or Poloxamer 407 or Poloxamer 188; and, at least 10% palmitic acid. In some preferred embodiments, the composition comprises at least about 10% Poloxamer 188.

In preferred embodiments, the first composition is administered within a blood vessel that is connected to a target. In preferred embodiments, the target is selected from the group consisting of a lesion, tumor, mass, nodule, arteriovenous malformation, or arteriovenous fistula. In preferred embodiments, the first composition is administered proximal to a target. In preferred embodiments, the target is selected from the group consisting of a lesion, tumor, mass, nodule, arteriovenous malformation, or arteriovenous fistula. In preferred embodiments, the first composition is administered in a collateral vessel of a target. In preferred embodiments, the first composition is delivered via syringe through a delivery device to a target. In preferred embodiments, the first composition comprises a solution and gel phase and is in the gel state upon administration. In preferred embodiments, the prevention of non-target embolization and/or diversion of blood in a mammal comprises administering a polymer to a blood vessel; wherein administration of the polymer occludes the vessel. In preferred embodiments, the administration is performed using a delivery device, more preferably a catheter or microcatheter, preferably wherein the microcatheter has a distal tip diameter between 1 French and 3 French.

In some embodiments, this disclosure provides methods for depositing a polymer in a blood vessel comprising the steps of: identifying a target within a mammal wherein the target comprises collateral vessels; navigating a delivery device to the collateral vessels; administering a polymer to the collateral vessel; and, performing a surgical step on the target. In preferred embodiments, the polymer is a thermosensitive polymer. In preferred embodiments, the polymer is delivered in a gel state and does not undergo a crosslinking mechanism. In preferred embodiments, the thermosensitive polymer is selected from the group consisting of poloxamine 1107, poloxamine 1307, poloxamer 338 poloxamer 407, and poloxamer 188. In preferred embodiments, the polymer has a transition temperature between 5° C. and 40° C. In preferred embodiments, the polymer comprises 5% to 40% by weight, preferably 20% to 35% by weight of said thermosensitive polymer. In preferred embodiments, the polymer also comprises 0.1% to 6% by weight of at least one excipient. In preferred embodiments, the excipient is selected from the group consisting of polyacrylic acid, carbomer, Carbopol 934P NF, carboxymethylcellulose, sodium hyaluronate, hydroxypropyl methylcellulose, alginate, sucrose, starch, or agar. In preferred embodiments, the polymer comprises more than about 50% by weight of phosphate buffered saline. In preferred embodiments, the target includes a lesion, tumor, mass, prostate, or uterine fibroid. In preferred embodiments, the surgical step includes TACE, TARE, DEB-TACE, administration of embolization particles, ablation, or administration of embolic material. In preferred embodiments, the polymer is capable of being instantaneously degraded via saline. In preferred embodiments, the saline is below 10° C. In preferred embodiments, the polymer is administered to multiple collateral vessels.

In some preferred embodiments, this disclosure provides methods for depositing a polymer in a blood vessel comprising the steps of: identifying a target within a mammal wherein the target comprises a vessel; navigating a delivery device to the vessel; administering a polymer to a portion of the vessel that is proximal to the target; optionally dissolving the polymer with saline; performing a surgical step on the target; and, optionally administering polymer after the surgical step. In preferred embodiments, the vessel is selected from the group consisting of the cystic artery, gastroduodenal artery, gastric artery, and collateral hepatic arteries.

Certain embodiments now being generally described, it will be more readily understood by reference to the following examples, which are included merely for purposes of illustration of certain aspects and embodiments of the present invention and are not intended to limit the disclosed embodiments.

### Example 1: Polymer Formulation and Degradation Testing

Poloxamers of the present invention were modified with various excipients to evaluate degradation time. The process for adding excipients includes: (1) Adding poloxamer and other solid additives (e.g., carboxymethylcellulose, hyaluronic acid, Carbopol, sodium hyaluronate, hydroxypropyl methylcellulose, and the like) to a jar wherein the mass of each component is weighed; (2) a stir bar is added to stir the dry powder ingredients; (3) with stirring, add the desired amount of solvent (e.g., 1× phosphate buffered saline (PBS), normal saline, or water), addition of water may minimize the formation of large clumps of material and result in faster dissolution; (4) the mixture is placed under refrigerated conditions, preferably 2° C.-5° C. until fully dissolved or homogenous; and, if necessary, (5) store the mixture at 2° C.-5° C. until ready for testing and/or use.

Polymers of this disclosure were screened through a degradation test, also referred to as a vial erosion test. To carry out this test: (1) a 5-gram amount of the polymer solution was loaded into a vial, if bubbles are visible the solution is cooled at a temperature between 2° C.-8° C. which will re-liquify the poloxamer solution (a stir bar may be added at this step to mix any more powder or dry excipients); (2) the solution was placed in an oven at 37° C. and allowed to form into a gel state; (3) 5 mL of 1×PBS at 37° C. was added on top of the gel layer within the vial; (4) the vials were attached to a rotisserie shaker within the 37° C. oven which rotates ˜7.5 rotations per minute (RPM); and, (5) every 30 minutes the PBS was decanted, the mass of the remaining gel measured, and 5 mL of PBS added on top of gel layer. The test ends when <10% of the gel mass is remaining or rather limit of quantification (LOQ). A summary of the tested polymer formulations and their respective degradations are shown in Table 1 below.

TABLE 1

| | Polymer Formulation | Time to <10% |
|---|---|---|
| Solution # | (wt %) | LOQ (hours) |
| 1 | Poloxamer 407 - 18% | 1.3 |
| | Poloxamer 188 - 8% | |
| | Saline - 0.9% | |
| 2 | Poloxamer 407 - 18% | N/A (degraded |
| | Poloxamer 188 - 8% | immediately) |
| | Saline - 5% | |
| 3 | Poloxamer 407 - 20% | 2.6 |
| | Carbopol 934P NF - 1% | |
| 4 | Poloxamer 188 - 25% | A (degraded |
| | Carbopol 934P NF - 1% | immediately) |
| 5 | Poloxamer 407 - 20% | 3.0 |
| | Carbopol 971P NF - 1% | |
| 6 | Poloxamer 188 - 25% | N/A (degraded |
| | Carbopol 971P NF - 1% | immediately) |
| 7 | Poloxamer 407 - 20% | 3.1 |
| | Noveon ® AA-1 - 1% | |
| 8 | Poloxamer 188 - 25% | N/A (degraded |
| | Noveon AA-1 - 1% | immediately) |
| 9 | Poloxamer 407 - 30% | 3.7 |
| | PBS 1x | |
| | Erioglaucine (1 mM) | |
| 10 | Poloxamer 407 - 25% | 2.0 |
| | Poloxamer 188 - 5% | |
| | PBS 1x | |
| 11 | Poloxamer 407 - 25% | 1.9 |
| | Poloxamer 188 - 15% | |
| | PBS 1x | |
| 12 | Poloxamer 407 - 30% | 4.2 |
| | Sodium Hyaluronate | |
| | 700 kDa - 0.5% | |
| 13 | Poloxamer 407 - 30% | 3.7 |
| | Sodium Hyaluronate | |
| | 700 kDa - 0.25% | |
| 14 | Poloxamer 407 - 19% | 2.6 |
| | Poloxamer 188 - 1% | |
| | Carbopol 934P NF - 1% | |
| | PBS 1x | |
| 15 | Poloxamer 407 - 19% | 6.2 |
| | Poloxamer 188 - 1% | |
| | Carbopol 934P NF - 2% | |
| | PBS 1x | |
| 16 | Poloxamer 407 - 19% | 10.6 |
| | Poloxamer 188 - 1% | |
| | Carbopol 934P NF - 4% | |
| | PBS 1x | |
| 17 | Poloxamer 407 - 19% | 1.3 |
| | Poloxamer 188 - 5% | |
| | CMC - 0.5% | |
| | PBS 1x | |
| 18 | Poloxamer 407 - 19% | 1.6 |
| | Poloxamer 188 - 5% | |
| | Carbopol 934P NF - 1% | |
| | CMC - 0.5% | |
| | PBX 1x | |
| 19 | Poloxamer 407 - 30% | 4.9 |
| | Sodium Hyaluronate | |
| | 700 kDa - 1% | |

It is further envisioned that the above polymers may be formulated with poloxamers including but not limited to Poloxamer: 237, 238, and 338. Further, the polymer may be formulated with a salt such as saline or wherein the salt is PBS. Further, the concentration range of Poloxamer 407 may range between (w/w %) 15%-45, or preferably 17.5%-27.5%, and most preferably 20%-22.5%. Further the viscosity modifier may include any combination of Carbopol, Carbopol 934P NF, Carbopol 971P NF, Noveon AA-1, CMC, Sodiuum Hyaluronate 1500 kDa, Sodium Hyaluronate 700 kDA, Alginate, Sucrose, Starch, and Agar.

A further formulation may include (all ingredients mention in (w/w %)): Saline (54.946%), Iohexol (25.0%), Tromethamine in Omnipaque 300 (0.05%), Edetate Calcium Diosodium (0.004%), and purified Poloxamer 407 (20.0%).

A further set of compositions were tested as discussed above for the compounds shown in Table 1. The results of the aggressive vial erosion test for these additional exemplary compositions are shown below in Table 2.

TABLE 2

| | Polymer Formulation | Time to <10% |
|---|---|---|
| Solution # | (wt %) | LOQ (hours) |
| 1 | Poloxamer 407 - 18% | 1.3 |
| | Poloxamer 188 - 8% | |
| | Saline - 0.9% | |
| 2 | Poloxamer 407 - 18% | N/A (degraded |
| | Poloxamer 188 - 8% | immediately) |
| | Saline - 5% | |
| 3 | Poloxamer 407 - 20% | 2.6 |
| | Carbopol 934P NF - 1% | |
| 4 | Poloxamer 188 - 25% | N/A (degraded |
| | Carbopol 934P NF - 1% | immediately) |
| 5 | Poloxamer 407 - 20% | 3.0 |
| | Carbopol 971P NF - 1% | |
| 6 | Poloxamer 188 - 25% | N/A (degraded |
| | Carbopol 971P NF - 1% | immediately) |
| 7 | Poloxamer 407 - 20% | 3.1 |
| | Noveon ® AA-1 - 1% | |
| 8 | Poloxamer 188 - 25% | N/A (degraded |
| | Noveon AA-1 - 1% | immediately) |
| 9 | Poloxamer 407 - 30% | 3.7 |
| | PBS 1x | |
| | Erioglaucine (1 mM) | |
| 10 | Poloxamer 407 - 25% | 2.0 |
| | Poloxamer 188 - 5% | |
| | PBS 1x | |
| 11 | Poloxamer 407 - 25% | 1.9 |
| | Poloxamer 188 - 15% | |
| | PBS 1x | |
| 12 | Poloxamer 407 - 30% | 4.2 |
| | Sodium Hyaluronate | |
| | 700 kDa - 0.5% | |
| 13 | Poloxamer 407 - 30% | 3.7 |
| | Sodium Hyaluronate | |
| | 700 kDa - 0.25% | |
| 14 | Poloxamer 407 - 19% | 2.6 |
| | Poloxamer 188 - 1% | |
| | Carbopol 934P NF - 1% | |
| | PBS 1x | |
| 15 | Poloxamer 407 - 19% | 6.2 |
| | Poloxamer 188 - 1% | |
| | Carbopol 934P NF - 2% | |
| | PBS 1x | |
| 16 | Poloxamer 407 - 19% | 10.6 |
| | Poloxamer 188 - 1% | |
| | Carbopol 934P NF - 4% | |
| | PBS 1x | |
| 17 | Poloxamer 407 - 19% | 1.3 |
| | Poloxamer 188 - 5% | |
| | CMC - 0.5% | |
| | PBS 1x | |
| 18 | Poloxamer 407 - 19% | 1.6 |
| | Poloxamer 188 - 5% | |
| | Carbopol 934P NF - 1% | |
| | CMC - 0.5% | |
| | PBS 1x | |
| 19 | Poloxamer 407 - 30% | 4.9 |
| | Sodium Hyaluronate | |
| | 700 kDa - 1% | |
| 20 | Poloxamer 407 - 20% | 3.2 |
| | Myristic Acid - 5% | |
| | PBS 1x | |
| 21 | Poloxamer 407 - 20% | 33.1 |
| | Myristic Acid - 10% | |
| | PBS 1x | |
| 22 | Poloxamer 407 - 18% | 24.3 |
| | Myristic Acid - 10% | |
| | PBS 1x | |
| 23 | Poloxamer 407 - 22% | 30.2 |
| | Myristic Acid - 10% | |
| | PBS 1x | |
| 24 | Poloxamer 407 - 20% | 84.7 |
| | Myristic Acid - 15% | |
| | PBS 1x | |
| 25 | Poloxamer 407 - 20% | 4.0 |
| | Capric Acid - 10% | |
| | PBS 1x | |
| 26 | Poloxamer 407 - 20% | 3.0 |
| | Linoleic Acid - 10% | |
| | PBS 1x | |
| 27 | Poloxamer 407 - 10% | 0.0 |
| | Myristic Acid - 10% | |
| | PBS 1x | |
| 28 | Poloxamer 407 - 10% | 41.8 |
| | Myristic Acid - 20% | |
| | PBS 1x | |
| 29 | Poloxamer 188 - 40% | 11.3 |
| | Myristic Acid - 10% | |
| | PBS 1x | |
| 30 | Poloxamer 188 - 30% | 3.4 |
| | Myristic Acid - 10% | |
| | PBS 1x | |
| 31 | Poloxamer 188 - 40% | 3.3 |
| | Stearic Acid - 10% | |
| | PBS 1x | |
| 32 | Poloxamer 188 - 30% | 32.4 |
| | Palmitic Acid - 10% | |
| | PBS 1x | |
| 33 | Poloxamer 188 - 30% | 9.2 |
| | Lauric Acid - 10% | |
| | PBS 1x | |
| 34 | Poloxamer 188 - 30% | 3.0 |
| | Linoleic Acid - 10% | |
| | PBS 1x | |
| 35 | Poloxamer 188 - 40% | 6.6 |
| | Lauric Acid - 10% | |
| | PBS 1x | |
| 36 | Poloxamer 188 - 40% | 4.5 |
| | Capric Acid - 10% | |
| | PBS 1x | |
| 37 | Poloxamer 407 - 18% | 42.9 |
| | Myristic Acid - 15% | |
| | PBS 1x | |

The data presented in Table 5 shows that most of the fatty-acids with higher melting points (e.g., myristic acid and palmitic acid) have increased degradation profiles as compared to lower melting point fatty acids (e.g., lauric, capric, and linoleic acids). Furthermore, increased degradation has been shown with multiple thermosensitive polymers including Poloxamer 407 (ethylene oxide-propylene oxide triblock copolymer, CAS Reg. No. 691397-13-4 (melting point 51-54° C.)) and Poloxamer 188 (CAS number 9003-11-6 (melting point about 52-57° C.). The data presented in Table 5 also shows that degradation time is directly correlated with the modulus of the composition (e.g., Solution #24 has a modulus of ˜120,000 Pa and Solution #37 has a modulus of ˜70,000 Pa).

It is also envisioned that the above polymer formulations may include a radiopaque modifier including metrizamide, iopamidol, iothalamate sodium, iohexol, iodomide sodium, gadolinium, and meglumine. Examples of water insoluble radiopaque materials include metals and metal oxides such as gold, titanium, silver, stainless steel, nitinol, oxides thereof, aluminum oxide, zirconium oxide, etc.

It is also envisioned that the polymer formulation can be spontaneously dissolved. Poloxamer is well known in the art to exhibit a sol-gel phase transition. For example, a 30% by weight poloxamer formulation undergoes a sol-gel transition at ˜10° C. It is therefore possible to return the gelled solution to a liquid upon cooling to a temperature below 10° C. One method to reversibly gel the polymer is to apply cold saline (<10° C.) to the gelled solution in-vivo which would return the polymer to the gel state and it would carried away due to physiologic blood flow.

While certain embodiments have been described in terms of the preferred embodiments, it is understood that variations and modifications will occur to those skilled in the art. Therefore, it is intended that the appended claims cover all such equivalent variations that come within the scope of the following claims.

1-59. (canceled)

60. A medicament for use in the prevention of non-target embolization in a mammal by administering a first composition comprising a thermosensitive polymer to a group consisting of hepatic vessels, prostatic vessels, and/or uterine vessels.

61. The medicament of claim 60, wherein said thermosensitive polymer is a block polymer, random copolymer, graft polymer or branched copolymer.

62. The medicament of claim 60 wherein the thermosensitive polymer is selected from the group consisting of poloxamine 1107, poloxamine 1307, poloxamer 338 poloxamer 407, and poloxamer 188.

63. The medicament of claim 60, wherein the thermosensitive polymer is poloxamer 407.

64. The medicament of claim 60, wherein the first composition further comprises at least one fatty acid, optionally wherein the fatty acid is selected from the group consisting of myristic acid, palmitic acid, stearic acid, linoleic acid, capric acid, lauric acid, and castor oil.

65. The medicament of claim 64, wherein the fatty acid is palmitic or myristic acid.

66. The medicament of claim 64, wherein the first composition comprises at least 10% w/w of the thermosensitive polymer Poloxamer 407 or Poloxamer 188 and at least 10% w/w of a fatty acid, optionally wherein the fatty acid is palmitic or myristic acid.

67. The medicament of claim 60, wherein the first composition comprises:
a transition temperature between 5° C. and 40° C.; and/or
5% to 40% by weight of the thermosensitive polymer; and/or
20% to 35% by weight of the thermosensitive polymer; and/or
0.1% to 6% by weight for each of at least one excipient, optionally wherein the excipient is selected from the group consisting of polyacrylic acid, carbomer, Carbopol 934P NF, carboxymethylcellulose, sodium hyaluronate, hydroxypropyl methylcellulose, alginate, sucrose, starch, and agar; and/or
more than about 50% by weight of phosphate buffered saline; and/or
a solution and gel phase and is in the gel phase upon administration.

68. The medicament of claim 60, wherein the first composition is formulated to be administered within a blood vessel that is connected to a target, optionally proximal to a target, and further optionally wherein the target is selected from the group consisting of a lesion, tumor, mass, nodule, arteriovenous malformation, or arteriovenous fistula.

69. The medicament of claim 60, wherein the first composition is formulated to be administered in a collateral vessel of a target.

70. The medicament of claim 60 contained within a delivery device.

71. The medicament of claim 60, wherein the delivery device is a catheter or microcatheter, optionally wherein the microcatheter comprises a distal tip diameter between 1 French and 3 French.

72. A method of depositing a polymer in a blood vessel comprising the steps of:
identifying a target within a mammal wherein the target comprises at least one collateral vessel;
navigating a delivery device to the at least one collateral vessel;
administering a polymer to the collateral vessel; and,
performing a surgical step on the target;
wherein:
the polymer is a thermosensitive polymer optionally selected from the group consisting of poloxamine 1107, poloxamine 1307, poloxamer 338 poloxamer 407, and poloxamer 188; and/or,
the polymer is delivered in a gel state and does not undergo a crosslinking reaction; and/or,
the polymer has a transition temperature between 5° C. and 40° C.; and/or,
the polymer comprises 5% to 40% by weight, preferably 20% to 35% by weight of said thermosensitive polymer; and/or,
the polymer also comprises 0.1% to 6% by weight of at least one excipient optionally selected from the group consisting of polyacrylic acid, carbomer, Carbopol 934P NF, carboxymethylcellulose, sodium hyaluronate, hydroxypropyl methylcellulose, alginate, sucrose, starch, or agar; and/or,
the polymer comprises more than about 50% by weight of phosphate buffered saline, optionally wherein the saline has a temperature of less than 10° C.; and/or,
the polymer is capable of being instantaneously degraded via saline; and/or,
the target includes a lesion, tumor, mass, prostate, or uterine fibroid; and/or,
the surgical step includes TACE, TARE, DEB-TACE, administration of embolization particles, ablation, or administration of embolic material; and/or,
the polymer is administered to multiple collateral vessels.

73. A method of depositing a polymer in a blood vessel comprising the steps of:
identifying a target within a mammal wherein the target comprises a vessel;
navigating a delivery device to the vessel;
administering a polymer to a portion of the vessel that is proximal to the target;
optionally dissolving the polymer with saline;
performing a surgical step on the target; and,
optionally administering polymer after the surgical step.

74. The method of claim 73, wherein the vessel is selected from the group consisting of the cystic artery, gastroduodenal artery, gastric artery, and collateral hepatic arteries.
